(19) United States
(12) Patent Application Publication
(10) Pub. No.: US 2025/0281226 A1
(43) Pub. Date: Sep. 11, 2025

(54) ELECTROSURGICAL SYSTEMS, DEVICES AND METHODS INCLUDING ECHOGENIC GUIDEWIRES

(71) Applicant: ElectroWire Corporation, Lexington, SC (US)

(72) Inventors: Robert Leonardi, Lexington, SC (US); Peter J. D'Aquanni, Murrieta, CA (US)

(21) Appl. No.: 19/075,881

(22) Filed: Mar. 11, 2025

Related U.S. Application Data
(60) Provisional application No. 63/563,667, filed on Mar. 11, 2024.

(51) Int. Cl. A61B 18/14; A61B 17/00; A61B 18/00

(52) U.S. Cl. CPC A61B 18/14; A61B 2017/00526; A61B 2018/00083; A61B 2018/00107; A61B 2018/00351; A61B 2018/00964

(57) ABSTRACT

An electrosurgical guidewire including a core wire. The core wire having a treated segment with an outer surface treated to impart an echogenic texture to the outer surface of the treated segment. The echogenic texture operating to increase ultrasound visibility of the treated segment.

### CROSS-REFERENCE TO RELATED APPLICATION

This application claims the benefit of U.S. Provisional Patent Application Ser. No. 63/563,667, filed Mar. 11, 2024 (pending), the disclosure of which is incorporated by reference herein in its entirety.

### TECHNICAL FIELD

The present disclosure generally relates to guidewires and associated core wires useful in electrosurgical procedures, and devices, systems and methods including such guidewires and associated core wires.

### BACKGROUND

Guidewires and their associated core wires are often constructed with differing physical characteristics along portions or segments of their length. The more distal tip segment or portion may include any of a wide variety of features that help the guidewire meet performance requirements. The more proximal segment or portion may have a “coil over core” design, wherein a relatively thin coil wire is wrapped repeatedly circumferentially around a thicker and stiffer core wire that is generally straight but flexible to allow its manipulation through the patient's body, such as through vasculature. The core wire may be thinner (i.e., formed with a smaller outer diameter) at the distal tip segment or portion of the guidewire and thicker at the more proximal shaft segment to meet performance requirements. Purposes of the thin coil wire include keeping a constant outer guidewire diameter (despite possibly variable core wire diameters) and to facilitate guidewire flexibility and passage through tortuous anatomies. Coil wires can have a secondary, often unintended benefit of being highly echogenic. In this regard, the thin coil wire structure creates a surface texture that helps ultrasound waves emitted by an ultrasound transducer to “echo” or reflect back to the ultrasound transducer in a way that generates a useful image on a display viewed by the doctor and other medical professionals performing a procedure.

Some guidewires, at least for most of the lengths of their shaft segments, do not have a coil covering the core. This “core-only” guidewire design maximizes wire stiffness at a given guidewire diameter because none of the guidewire diameter is “lost” to flexible wraps of coil wire. Core-only guidewire designs are used to maximize guidewire stiffness within the constraints of a maximum outer diameter.

Electrosurgical guidewires are unique in that they require electrical insulation. Electrosurgical guidewires are not unique in that they may also need to be relatively stiff at small diameters. Core-only guidewire designs are typical for electrosurgical guidewire bodies because some of the “available” guidewire diameter is necessarily “lost” to the required insulation. Since adequate core wire insulation and stiffness are relatively more important, the coil wire may be eliminated along with its echogenicity.

Electrosurgical wire-based puncture of the interatrial septum is now commonly used for transseptal procedures, which may involve over-the-wire and/or next-to-the-wire delivery of devices to the left atrium. Next-to-the-wire delivery of devices to the left atrium is facilitated by echocardiographic visualization of the transseptal guidewire passing through the interatrial septostomy. In left atrial arrhythmia ablation procedures, for example, such visualization may be needed to facilitate passage of mapping and/or ablation catheters alongside the transseptal guidewire through the same interatrial septostomy. Similarly, when left atrial appendage occlusion procedures or mitral valve interventions are guided by intracardiac echocardiography (ICE), such visualization may be needed to facilitate passage of an ICE catheter alongside the transseptal guidewire through the same septostomy. If the transseptal guidewire is poorly echogenic, poor echocardiographic visualization of the transseptal guidewire causes difficulty advancing devices alongside the transseptal guidewire. If the procedure is a fluoroscopy-free one, the difficulty of advancing devices may be more significant, and there may be little recourse.

Currently available electrosurgical guidewires have core-only shaft segments that, within the constraints of a maximum outer diameter, optimize guidewire insulation and stiffness at the expense of echogenicity. It would be beneficial to provide electrosurgical guidewires and associated devices, systems and methods that, within the constraints of a maximum outer diameter, provide improved visualization through echogenicity while still providing adequate electrical insulation and stiffness.

### SUMMARY

In one or more general aspects, electrosurgical guidewires are provided. One such guidewire includes a core wire having a segment with an outer surface treated to impart an echogenic texture to that segment. This makes that segment distinctly echogenic and therefore distinctly visible by an imaging system relative to an adjacent segment or segments of the wire. Particularly, the echogenic texture increases distinct ultrasound visibility of the treated segment of the wire.

The electrosurgical guidewire may have one or more optional and/or additional features. Several examples, which may provide one or more distinct benefits themselves or in any of various combinations are discussed herein. For instance, more than one segment of the outer surface may be treated to impart the echogenic texture. The outer surface may be treated without removal of a significant amount of core wire material and, therefore, without significant compromise of wire stiffness at the treated segment(s). The treatment methods may, for example, include one or more surface treatments such as bead blasting, dimpling, peening, grinding, laser treatment, sanding, or any other treatments or combinations of treatments that create echogenic irregularity of the wire. The outer surface may be covered by at least one of: spray coating, shrink tubing, or other electrical insulation materials. The wire may be used in transseptal procedures, and the treated segment may then further comprise a segment that lies across the interatrial septostomy after transseptal puncture. The length of the treated segment may be between about 1 cm and about 10 cm. The maximum outer diameter of the guidewire may be about 0.014 inches to 0.038 inches. The treated segment may further comprise circumferentially applied texturing treatment for creating echogenic irregularity of the outer surface that is oriented transverse to an ultrasound transducer. This orientation can further increase echogenicity and, therefore, optimize visualization. The electrosurgical guidewire may include a coiled wire wrapped around a portion of the core wire. In some embodiments, the coiled wire may be wrapped around a portion of the treated segment.

An electrosurgical system may include the electrosurgical guidewire and an apparatus for coupling the electrosurgical guidewire to an electrosurgical generator. The apparatus may include an elongated flexible conductive element, an activator unit for selectively controlling energy to the electrosurgical guidewire, and a coupler for removably coupling the electrosurgical guidewire to the apparatus. The electrosurgical system may include an electrosurgical generator.

In additional or alternative aspects, methods of using an electrosurgical guidewire are provided and generally comprise directing an electrosurgical guidewire into a patient. The electrosurgical guidewire includes a core wire having a treated segment with an outer surface treated to impart an echogenic texture thereto, such as in any of the broader and/or specific manners discussed herein. The echogenic texture increases ultrasound visibility of the treated segment. The method includes visualizing the treated segment using an ultrasonic imaging system.

The methods may include, for example, one or more of the optional features discussed herein. Illustrative examples are discussed below, but of course other features or options may be included instead of or in addition to those specifically discussed herein. More than one segment of the outer surface may be treated to impart the echogenic texture, and visualizing the segment may further comprise visualizing more than one segment. The outer surface may be treated without removal of a significant amount of wire material and without significant compromise of wire stiffness. The outer surface may be covered by at least one of: spray coating, shrink tubing, or other electrical insulation materials. The outer surface may be surface treated by at least one of: bead blasting, dimpling, peening, grinding, laser treatment, sanding, or any of these and/or other treatments or combinations of treatments that create echogenic irregularity of the wire.

The method may further comprise a transseptal procedure, and in this case may further comprise visualizing the segment lying across the interatrial septostomy after transseptal puncture. The length of the segment may be between about 1 cm and about 10 cm. The maximum outer diameter of the guidewire may be about 0.014 inches to 0.038 inches. The treated segment may further comprise circumferentially applied texturing treatment for creating echogenic irregularity of the outer surface wherein the surface treatment is oriented transverse to an ultrasound transducer, and visualizing the segment further comprises visualizing the transversely oriented echogenic irregularity. In some embodiments, the electrosurgical guidewire may include a coiled wire wrapped around a portion of the core wire. The method may include visualizing the coiled wire. The coiled wire may be wrapped around a portion of the treated segment.

In some embodiments of the method, the electrosurgical guidewire may be part of an electrosurgical system. The electrosurgical system may include the electrosurgical guidewire and an apparatus for coupling the electrosurgical guidewire to an electrosurgical generator. The apparatus may include an elongated flexible conductive element, an activator unit for selectively controlling energy to the electrosurgical guidewire, and a coupler for removably coupling the electrosurgical guidewire to the apparatus. The electrosurgical system may also include an electrosurgical generator.

Additional and/or alternative aspects of the invention may include systems that include guidewires within the scope of this invention. As an example, such a system may include a radiofrequency (RF) energy generation unit useful in supplying RF energy to the guidewire so that the tip can easily penetrate tissue such as septal tissue of the heart. Additional system components may include visualization equipment/devices used during such procedures to provide one or more images of the wire segment(s) during a medical procedure.

Various additional features and advantages will become readily apparent to those of ordinary skill in the art upon review of the following detailed description of the illustrative embodiments. Any of the features and functions described herein may be applied to any of the disclosed embodiments or methods.

### BRIEF DESCRIPTION OF THE DRAWINGS

FIG. 1 shows an illustrative electrosurgical transseptal puncture guidewire.

FIG. 1A is a perspective view of an illustrative system for delivering RF energy to tissue during a medical procedure including the electrosurgical guidewire of FIG. 1.

FIG. 2 is a cross sectional view taken along line 2-2 of FIG. 1.

FIG. 3 shows an alternative illustrative electrosurgical transseptal puncture guidewire.

FIG. 4 illustrates a heart model with an electrosurgical transseptal puncture guidewire and an intracardiac echocardiography catheter inserted into the model.

FIG. 5A is an intracardiac echocardiography image of an electrosurgical transseptal puncture guidewire with an untreated guidewire core.

FIG. 5B is an intracardiac echocardiography image of an electrosurgical transseptal puncture guidewire with a segment treated to create an echogenic texture operating to increase the ultrasound visibility of the treated segment.

### DETAILED DESCRIPTION OF ILLUSTRATIVE EMBODIMENTS

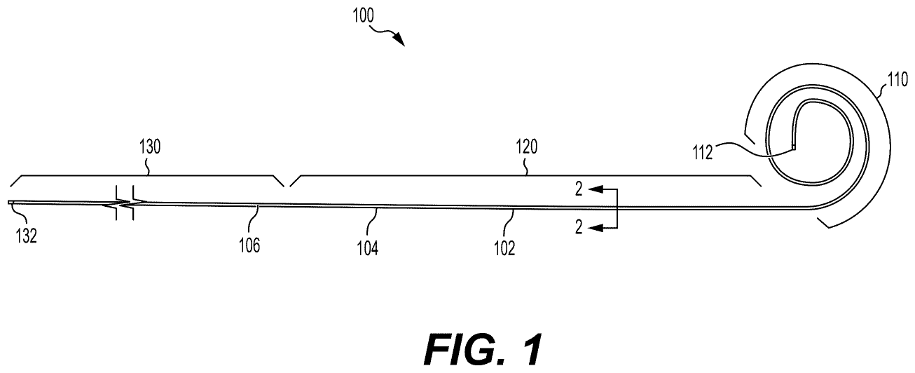

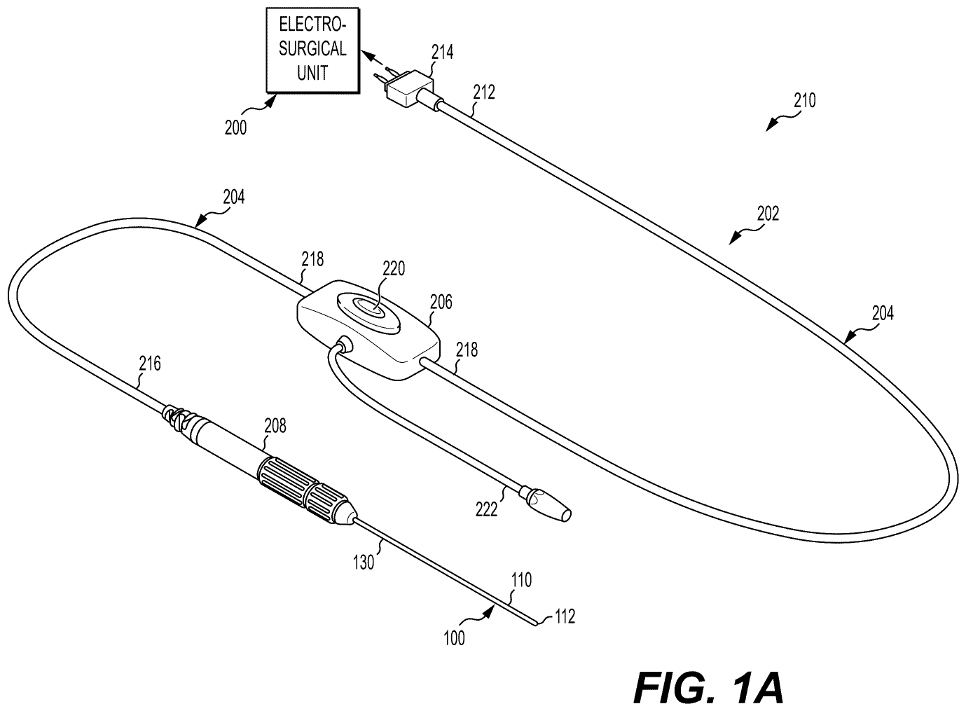

FIG. 1 shows an illustrative electrosurgical transseptal puncture guidewire 100 and FIG. 2 illustrates a cross section of the electrosurgical guidewire 100. The electrosurgical guidewire 100 includes a core wire 102 with an outer surface 104 covered by electrical insulation 106. In an illustrative aspect, the electrosurgical guidewire 100 may be of any desired design in terms of being, for example, “core-only” or “coil-over-core.” For convenience only, this detailed description uses the term “core,” but the disclosure herein is equally applicable, as will become apparent to those of ordinary skill, to “core-only” segments of coil-over core guidewire designs.

The electrosurgical guidewire 100 includes a distal wire segment 110, a more proximal wire segment 120, and a shaft segment 130. The distal wire segment 110 includes a bare or exposed distal tip 112 or terminus of the electrosurgical guidewire 100 serving as the “active electrode” in the system, where electric current density is highly concentrated to effect tissue vaporization. The electrosurgical guidewire 100 depicted in FIG. 1 includes a pigtail shaped distal wire segment 110, but a J-tipped, straight-tipped, or other design configuration may be used. The shaft segment 130 has a bare or exposed portion 132 for purposes of electrical connection to, for example, an electrosurgical generator unit supplying radiofrequency (RF) energy. The electrosurgical guidewire 100 together with an electrosurgical generator unit, along with any other desired components useful during medical procedures involving the use of the electrosurgical guidewire 100 and electrosurgical generator unit, can comprise a system in accordance with various embodiments. In some embodiments, these bare or exposed portions 112, 132 of the core wire 102 may be located at respective distal and proximal terminus points or ends. However, in other embodiments, for example, one or both of these bare or exposed portions 112, 132 may be located near but not at a terminus point. In some embodiments, a bare or exposed portion, serving as an active electrode, may be located remotely from the terminus points or ends. The remainder of the electrosurgical guidewire 100 must be insulated to minimize charge dispersion and related loss of electrosurgical effect.

In this illustrative embodiment, the outer surface 104 of the core wire 102 of the more proximal wire segment 120 is treated to impart an echogenic texture to the more proximal wire segment 120. The echogenic texture increases the visibility by an imaging device such as an ultrasound-based imaging device. The treatment of the surface 104 of the core wire 102 may be usefully applied to any core-only segment(s) of the electrosurgical guidewire 100, including more distal wire segments 110, more proximal wire segments 120, and/or shaft segments 130. The outer diameter of the core wire 102 is an important determinant of mechanical guidewire performance (e.g. mechanical characteristics such as strength and flexibility). The treatment of the surface 104 of the core wire 102 may be completed without removal of a significant amount of core wire material and without significant compromise of core wire stiffness. Options for segmental treatment of the surface 104 of the core wire 102 may include bead blasting, dimpling, peening, grinding, laser treatment, sanding, and any other treatments or combinations of treatments that create echogenic irregularity in the otherwise smooth surface of the core wire 102. The insulation 106 is the main determinant of electrosurgical guidewire performance. Treated segments of the core wire 102 may or may not be covered by spray coating, shrink tubing, or other electrical insulation materials.

FIG. 1A is a perspective view of an illustrative system for delivering RF energy to tissue during a medical procedure. Further illustrative details of this exemplary system may be found in U.S. patent application Ser. No. 18/243,927, filed on Sep. 8, 2023, the disclosure of which is hereby incorporated by reference herein. In this illustrative embodiment, the electrosurgical guidewire 100, an electrosurgical unit 200, and an apparatus 202 including an elongated flexible conductive element 204, an activator unit 206, and a coupler 208 form the system 210 which is configured for performing a medical procedure. The elongated flexible conductive element 204 is an electrically insulated conductor, such as a wire, configured for transmitting electricity or RF energy. In some embodiments, the elongated flexible conductive element 204 may be a cable including an insulated wire or wires and having a protective casing. The elongated flexible conductive element 204 includes a proximal end 212 including an electrosurgical unit connector 214, and a distal end 216 coupled to the coupler 208. In this illustrative embodiment, the coupler 208 is configured to removably couple the electrosurgical guidewire 100 to the elongated flexible conductive element 204. The electrosurgical unit connector 214, capable of attaching to conventional RF energy generating units for delivering RF energy, may releasably connect to the electrosurgical unit 200. Many commercially available electrosurgical units include a standardized receptacle, such as a monopolar accessory receptacle. The electrosurgical unit connector 214 may be configured to couple to any one of a plurality of electrosurgical units with standardized receptacles. Therefore, the subsystem or assembly comprising, for example, the electrosurgical guidewire 100, and the apparatus 202 including the elongated flexible conductive element 204, activator unit 206, and coupler 208 may be physically coupled to one of several different standardized receptacles of an electrosurgical RF generating unit. In the illustrative embodiment the elongated flexible conductive element 204 has a mid-portion 218 that is connected to the activator unit 206. The activator unit 206 is situated at a location spatially separated from the proximal end 212 of the elongated flexible conductive element 204 and, therefore also spatially separated from the electrosurgical unit 200, by a segment of the elongated flexible conductive element 204. The activator unit 206 also may be spatially separated from the coupler 208 by another segment of the elongated flexible element 204, as shown in FIG. 1A or, optionally, the activator unit 206 may be integrated with or otherwise fixed to the coupler 208. In some embodiments, the activator unit 206 may be located at the proximal end 212 of the elongated flexible conductive element 204 adjacent to or otherwise fixed to the electrosurgical unit connector 214. The activator unit 206 includes a switch element 220, such as a push button or other element, allowing the user to selectively activate the flow of RF energy. The apparatus 202 includes an electroanatomical mapping connector 222 that is capable of connecting to electroanatomical mapping systems.

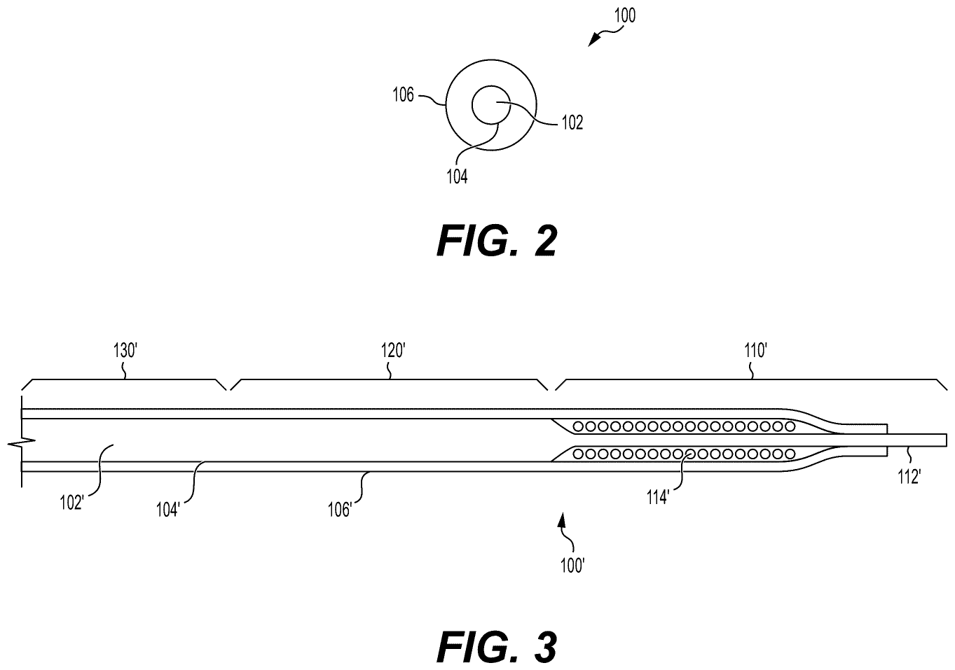

FIG. 3 shows an alternative illustrative electrosurgical transseptal puncture guidewire 100′. Generally, the electrosurgical guidewire 100′ is similar in construction and operation to the electrosurgical guidewire 100 described above and the electrosurgical guidewire 100′ may be substituted for other electrosurgical guidewires, or any electrosurgical guidewire 100′ may be used, in various other exemplary embodiments according to the present disclosure. Like reference numbers refer to like components. For brevity, the following description minimizes redundant description and focuses on the differences between the electrosurgical guidewire 100 and the electrosurgical guidewire 100′.

The electrosurgical guidewire 100′ includes a core wire 102′ with an outer surface 104′ covered by electrical insulation 106′. The electrosurgical guidewire 100′ includes a distal wire segment 110′, a more proximal wire segment 120′, and a shaft segment 130′. The distal wire segment 110′ includes a bare or exposed distal tip 112′ or terminus of the electrosurgical guidewire 100′ serving as the “active electrode” in the system. The electrosurgical guidewire 100′ depicted in FIG. 3 includes a straight distal wire segment 110′, but a pigtail shaped, J-tipped, or other design configuration may be used.

In this illustrative embodiment, distal wire segment 110′ includes a coiled wire 114′ located over the core wire 102′. At this position, the coiled wire 114′ imparts an echogenic texture to the portion of the distal wire segment 110′ covered by the coiled wire 114′. The coiled wire 114′ increases the ultrasound visibility of the portion of the distal wire segment 110′ covered by the coiled wire 114′. For example, like the electrosurgical guidewire 100, this coiled wire segment may provide a distinct segment of increased echogenicity relative to one or more adjacent portions of the electrosurgical guidewire 100′. This may provide a targeted visualization of that enhanced echogenic segment relative to the one or more adjacent segments. Any portion of the distal wire segment 110′, including the portion covered by the coiled wire 144′, may be treated to impart an echogenic texture.

The outer surface of the core wire of the more proximal wire segment 120′ is treated to impart an echogenic texture to the more proximal wire segment 120′. The treatment of the surface of the core wire may be usefully applied to any core-only segment(s) of the electrosurgical guidewire 100′, including more distal wire segments 110′, more proximal wire segments 120′, and/or shaft segments 130′.

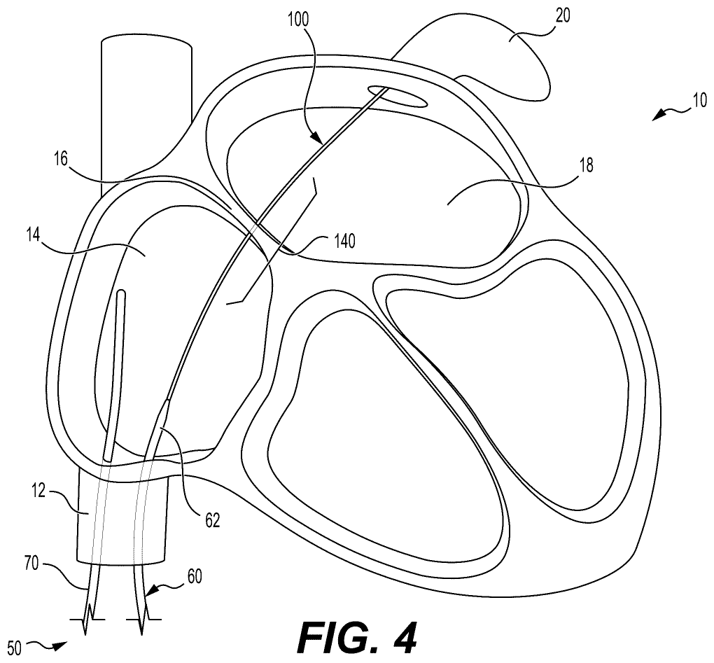

FIG. 4 illustrates a water-filled heart model 10 useful in benchtop assessment of the echogenicity (and echocardiographic visibility) of transseptal guidewires. Illustrated is an introducer set 60 extending from the inferior vena cava 12 into the right atrium 14 with the electrosurgical guidewire 100 extending from the tip of a dilator 62 of the introducer set 60, through the interatrial septum 16, into the left atrium 18, and, in this case, into the left atrial appendage 20. Also shown is an ICE catheter 70 extending from the inferior vena cava 12 into the right atrium 14. For electrosurgical transseptal puncture guidewires that are used in transseptal procedures, the segment 140 of the electrosurgical guidewire 100 that lies across the interatrial septum 16 after transseptal puncture is the most clinically relevant segment.

Depending on the size of the left atrium 18 (which determines how much wire might be advanced into the left atrium 18 after transseptal puncture), the relevant segment 140 may ideally be about 6 cm long, and a preferred but exemplary range of the relevant segment length is about 1 cm to about 10 cm. For electrosurgical transseptal puncture guidewires 100 used in transseptal procedures, the maximum outer diameter of the guidewire 100 may be constrained to less than about 0.014 to 0.038 inches to maintain compatibility with available introducer sets.

Since the long axes of transseptal guidewires and right atrial ICE catheters are roughly parallel in the heart when both devices are inserted from a femoral vein, circumferentially applied texturing treatment (analogous to wraps of coil wire around a core wire) may create echogenic irregularity of the core wire's surface that is generally orthogonal to the ICE catheter's ultrasound transducer. Other transverse orientations may be used instead. The circumferentially applied, transversely oriented texturing treatment may increase echogenicity (and echocardiographic visibility) more substantially than a longitudinally applied, or less transversely oriented texturing treatment.

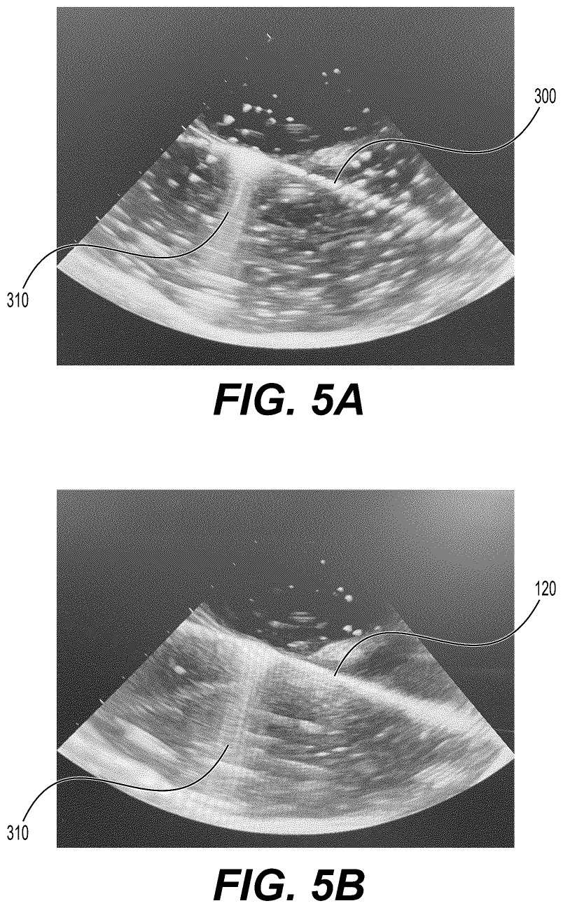

A water-filled heart model 10, illustrated in FIG. 4, was used to obtain the intracardiac echocardiography images in FIGS. 5A and 5B. The intracardiac echocardiography image in FIG. 5A was obtained using an untreated guidewire 300 having a core wire with a relatively smooth surface. The intracardiac echocardiography image in FIG. 5B was obtained using an illustrative embodiment of an electrosurgical transseptal puncture guidewire with a segment 120 of the core wire treated to create an echogenic texture operating to increase the ultrasound visibility of the treated segment. The guidewire in FIG. 5B is much more echodense (easier to see) than the guidewire 300 in FIG. 5A. The echogenic texture on the illustrative guidewire embodiment used in FIG. 5B was created by circumferentially “sanding” the guidewire core by using a drill motor to spin it between opposed pieces of 80-grit sandpaper. The sanding process imparted a palpable texture to the wire's surface but did not remove a significant volume of material from the wire. The circumferentially sanded guidewire core depicted in FIG. 5B is substantially more echogenic (and echocardiographically visible) than the untreated guidewire core depicted in FIG. 5A. It should be understood that this is just one illustrative example of a treatment applied to a core wire to create an echogenic texture and other treatments may be applied to a core wire to generate the disclosed echogenic texture. It should be noted that the images of FIGS. 5A and 5B include reverberation artifacts 310 which is a type of ultrasound imaging artifact that occurs when sound waves bounce back and forth between two highly reflective surfaces, creating multiple echoes. These reverberation artifacts 310 do not represent any structure of the heart model or guidewires.

While the present invention has been illustrated by the description of specific embodiments thereof, and while the embodiments have been described in considerable detail, it is not intended to restrict or in any way limit the scope of the appended claims to such detail. The various features discussed herein may be used alone or in any combination within and between the various embodiments. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope or spirit of the general inventive concept.

What is claimed is:

1. An electrosurgical guidewire, comprising:
a core wire having a treated segment with an outer surface treated to impart an echogenic texture thereto, the echogenic texture operating to increase ultrasound visibility of the treated segment.

2. The electrosurgical guidewire of claim 1, wherein the core wire comprises more than one treated segment.

3. The electrosurgical guidewire of claim 1, wherein the outer surface of the treated segment is treated without removal of a significant amount of wire material and without significant compromise of wire stiffness.

4. The electrosurgical guidewire of claim 1, further comprising a coating of at least one of: spray coating, shrink tubing, or other electrical insulation materials.

5. The electrosurgical guidewire of claim 1, wherein the outer surface of the treated segment is treated by at least one of: bead blasting, dimpling, peening, grinding, laser treatment, sanding, or any other treatments or combinations of treatments that create echogenic irregularity of the treated segment.

6. The electrosurgical guidewire of claim 1, wherein the core wire is used in transseptal procedures, and a portion of the treated segment lies across the interatrial septostomy after transseptal puncture.

7. The electrosurgical guidewire of claim 6, wherein the length of the treated segment is between about 1 cm and about 10 cm.

8. The electrosurgical guidewire of claim 1, wherein the maximum outer diameter of the guidewire is about 0.014 inches to 0.038 inches.

9. The electrosurgical guidewire of claim 1, wherein the treated segment further comprises circumferentially applied texturing treatment for creating echogenic irregularity of the outer surface that is oriented transverse to an ultrasound transducer.

10. The electrosurgical guidewire of claim 1, further comprising a coiled wire wrapped around a portion of the core wire.

11. The electrosurgical guidewire of claim 10, wherein the coiled wire is wrapped around a portion of the treated segment.

12. An electrosurgical system comprising:
the electrosurgical guidewire of claim 1; and
an apparatus for coupling the electrosurgical guidewire to an electrosurgical generator, the apparatus including:
an elongated flexible conductive element,
an activator unit for selectively controlling energy to the electrosurgical guidewire, and
a coupler for removably coupling the electrosurgical guidewire to the apparatus.

13. The electrosurgical system of claim 12, further comprising an electrosurgical generator.

14. A method of using an electrosurgical guidewire, comprising:
directing an electrosurgical guidewire into a patient, the electrosurgical guidewire including a core wire having a treated segment with an outer surface treated to impart an echogenic texture thereto, the echogenic texture operating to increase ultrasound visibility of the treated segment, and
visualizing the treated segment using an ultrasonic imaging system.

15. The method of claim 14, wherein the core wire includes more than one treated segment, and visualizing the treated segment further comprises visualizing more than one treated segment.

16. The method of claim 14, wherein the outer surface of the treated segment is treated without removal of a significant amount of wire material and without significant compromise of wire stiffness.

17. The method of claim 14, wherein the electrosurgical guidewire includes a coating of at least one of: spray coating, shrink tubing, or other electrical insulation materials.

18. The method of claim 14, wherein the outer surface of the treated segment is treated by at least one of: bead blasting, dimpling, peening, grinding, laser treatment, sanding, or any other treatments or combinations of treatments that create echogenic irregularity of the treated segment.

19. The method of claim 14, wherein the method further comprises a transseptal procedure, and visualizing the treated segment further comprises visualizing the treated segment lying across the interatrial septostomy after transseptal puncture.

20. The method of claim 19, wherein the length of the treated segment is between about 1 cm and about 10 cm.

21. The method of claim 14, wherein the maximum outer diameter of the guidewire is about 0.014 inches to 0.038 inches.

22. The method of claim 14, wherein the treated segment further comprises circumferentially applied texturing treatment for creating echogenic irregularity of the outer surface that is oriented transverse to an ultrasound transducer, and visualizing the treated segment further comprises visualizing the transversely oriented echogenic irregularity.

23. The method of claim 14, wherein the electrosurgical guidewire further comprises a coiled wire wrapped around a portion of the core wire, and
the method further comprises visualizing the coiled wire.

24. The method of claim 23, wherein the coiled wire is wrapped around a portion of the treated segment.

25. The method of claim 14, wherein the electrosurgical guidewire is part of an electrosurgical system comprising the electrosurgical guidewire and an apparatus for coupling the electrosurgical guidewire to an electrosurgical generator, the apparatus includes an elongated flexible conductive element, an activator unit for selectively controlling energy to the electrosurgical guidewire, and a coupler for removably coupling the electrosurgical guidewire to the apparatus.

26. The method of claim 25, wherein the electrosurgical system further comprises an electrosurgical generator.
